(19) United States
(12) Patent Application Publication
(10) Pub. No.: US 2023/0066994 A1
(43) Pub. Date: Mar. 2, 2023

(54) INFORMATION PROCESSING SYSTEM, PROGRAM, AND INFORMATION PROCESSING METHOD

(71) Applicant: SoftBank Corp., Tokyo (JP)

(72) Inventors: Yoshihiro ENDO, Tokyo (JP); Koji HIDAKA, Kanagawa (JP); Naohiro AKIYAMA, Ibaraki (JP); Masanori TANZAWA, Kanagawa (JP); Junichiro MAMIYA, Saitama (JP)

(21) Appl. No.: 17/982,446

(22) Filed: Nov. 7, 2022

Related U.S. Application Data
(63) Continuation of application No. PCT/JP2021/032940, filed on Sep. 8, 2021.

(30) Foreign Application Priority Data
Sep. 10, 2020 (JP) 2020-152408

(51) Int. Cl. G16H 50/80; G16H 40/20

(52) U.S. Cl. CPC G16H 50/80; G16H 40/20

(57) ABSTRACT

Provided is an information processing system including: a target person information reception unit configured to receive target person information including information related to a state of a target person and a situation in a periphery of the target person from an image capturing apparatus; a target person information analysis unit configured to analyze the target person information; and a notification processing execution unit configured to execute notification processing based on an analysis result obtained by the target person information analysis unit. Provided is an information processing method executed by a computer, including: receiving target person information including information related to a state of a target person and a situation in a periphery of the target person from an image capturing apparatus; analyzing the target person information; and executing notification processing based on an analysis result obtained in the analyzing of the target person information.

The contents of the following Japanese patent application(s) are incorporated herein by reference:
NO. 2020-152408 filed in JP on Sep. 10, 2020
NO. PCT/JP2021/032940 filed in WO on Sep. 8, 2021

### BACKGROUND

### 1. Technical Field

The present invention relates to an information processing system, a program, and an information processing method.

### 2. Related Art

Patent Document 1 describes an AI camera that recognizes an object present in a predetermined shooting area (see, for example, Patent Document 1).

### PRIOR ART DOCUMENT

### Patent Document

Patent Document 1: Japanese Patent Application Publication No. 2020-052484

### BRIEF DESCRIPTION OF THE DRAWINGS

FIG. 1 schematically shows an example of a system 10.

FIG. 2 schematically shows an example of the system 10.

FIG. 3 schematically shows an example of the system 10.

FIG. 4 schematically shows an example of a functional configuration of an information processing system 100.

FIG. 5 schematically shows an example of a hardware configuration of a computer 1200 that functions as the information processing system 100.

### DESCRIPTION OF EXEMPLARY EMBODIMENTS

Hereinafter, the present invention will be described through an embodiment of the invention, but the following embodiment does not limit the invention according to the claims. In addition, not all of the combinations of features described in the embodiment are essential to the solving means of the invention.

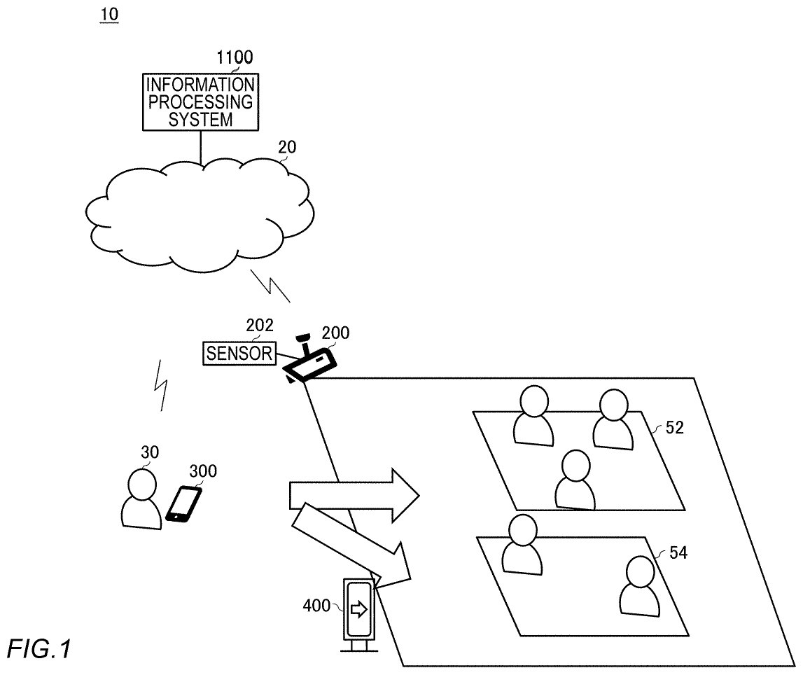

FIG. 1 schematically shows an example of a system 10. The system 10 includes an information processing system 100. The information processing system 100 may be realized on a cloud. The information processing system 100 may be realized by a single apparatus. In other words, the information processing system 100 may be an information processing apparatus. The information processing system 100 may be realized by a plurality of apparatuses. The system 10 may include an image capturing apparatus 200. The system 10 may also include a display apparatus 400.

The system 10 according to the present embodiment acquires, by the image capturing apparatus 200, target person information including information related to a state of a target person 30 and a peripheral situation of the target person 30, and executes notification processing that is based on an analysis result obtained by analyzing the target person information. The system 10 may be used for guiding a plurality of target persons 30 to respective movement destinations suited to them. The system 10 may be applicable to various fields.

The system 10 is applied to, for example, medical settings. As a specific example, the system 10 guides a target person 30 who has visited a fever clinic placed outside to a movement destination corresponding to a state of the target person 30. Further, as a specific example, the system 10 guides a target person 30 who has come to a waiting lounge of a hospital or the like to a movement destination depending on a state of the target person 30. Furthermore, as a specific example, the system 10 guides a target person 30 who has visited an isolation facility for isolating patients infected with a disease to a movement destination corresponding to a state of the target person 30. In addition, the system 10 may also be applied to so-called drive-through testing. The system 10 determines a state of a target person 30 who visits a testing site by a vehicle, and guides the target person 30 to a movement destination corresponding to the state of the target person 30, or registers the state of the target person 30 in association with the vehicle that the target person 30 is on, and notifies an administrator or the like.

The system 10 may be applied to any application without limitation to medical settings. For example, the system 10 is applied to facilities such as a school, kindergarten, and nursery school. For example, the system 10 determines a state of a student who has come to school, and when there is a problem in coming, notifies that student, a guardian of that student, a teacher relevant to that student, and the like to that effect. Alternatively, for example, the system 10 determines a state of a child who has come to a kindergarten or nursery school, and when there is a problem in coming, notifies a guardian of that child, a teacher relevant to that child, and the like to that effect. Alternatively, for example, the system 10 determines a state of a child staying at a kindergarten or nursery school, and when there is a problem in staying, notifies a guardian of that child, a teacher relevant to that child, and the like to that effect.

The system 10 may be applied to a retail store and the like. For example, the system 10 guides a target person 30 who has visited a retail store to a movement destination corresponding to a state of the target person 30. The system 10 may be applied to any other applications.

In FIG. 1, descriptions will be given while mainly taking a case of targeting a fever clinic as an example. The image capturing apparatus 200 captures a target person 30 who has visited a fever clinic and a periphery of the target person 30.

The image capturing apparatus 200 may determine a peripheral situation of the target person 30 by analyzing the captured image. The image capturing apparatus 200 determines a situation of people in a periphery of the target person 30, for example. The image capturing apparatus 200 may determine the number of people in the periphery of the target person 30. The image capturing apparatus 200 may determine a positional relationship between the target person 30 and the people in the periphery of the target person 30.

The image capturing apparatus 200 may determine a state of the target person 30 by analyzing the captured image. The image capturing apparatus 200 determines a body motion of the target person 30, for example. The image capturing apparatus 200 determines a level of breathing of the target person 30, for example. The image capturing apparatus 200 determines a breathing speed or roughness of the breathing of the target person 30, for example. The image capturing apparatus 200 may also determine whether the target person 30 is holding any part of a body, or the like, for example.

The image capturing apparatus 200 may acquire, from the sensor 202, a peripheral situation of the target person 30 detected by a sensor 202. The sensor 202 may include one or more sensors. The sensor 202 includes, for example, a carbon dioxide sensor that measures a carbon dioxide concentration in the periphery of the target person 30. The image capturing apparatus 200 may acquire, from the sensor 202, the carbon dioxide concentration in the periphery of the target person 30. The sensor 202 includes, for example, an air volume sensor that measures an air volume in the periphery of the target person 30. The image capturing apparatus 200 may acquire the air volume in the periphery of the target person 30 from the sensor 202.

The image capturing apparatus 200 may acquire the state of the target person 30, that has been detected by the sensor 202, from the sensor 202. The sensor 202 includes, for example, a body temperature sensor that measures a body temperature of the target person 30. The body temperature sensor may be any sensor as long as it can detect a body temperature of the target person 30. The body temperature sensor may detect the body temperature of the target person 30 in a non-contact manner. The body temperature sensor may be, for example, an infrared temperature sensor. The body temperature sensor may be a sensor that measures the body temperature of the target person 30 while being contact with the target person 30.

The information processing system 100 receives target person information including information related to a state of the target person 30 and a peripheral situation of the target person 30 from the image capturing apparatus 200 that has acquired the target person information. The information processing system 100 receives the target person information from the image capturing apparatus 200 via a network 20.

The network 20 includes a cloud. The network 20 may include a mobile communication network. The mobile communication network may conform to any of an LTE (Long Term Evolution) communication system, a 3G (3rd Generation) communication system, a 5G (5th Generation) communication system, and a 6G (6th Generation) communication system and subsequent communication systems.

The information processing system 100 may be connected to the network 20 by wires. The information processing system 100 may alternatively be wirelessly connected to the network 20. The image capturing apparatus 200 may be connected to the network 20 by wires. The image capturing apparatus 200 may alternatively be wirelessly connected to the network 20. The wireless connection may be realized via a wireless base station, a Wi-Fi (registered trademark) access point, and the like.

The information processing system 100 analyzes the target person information received from the image capturing apparatus 200, and executes notification processing that is based on the analysis result. The information processing system 100 may execute notification processing for notifying the target person 30 of a content that differs between a case where the body temperature of the target person 30 is higher than a predetermined threshold and a case where the body temperature is lower than the threshold. For example, the information processing system 100 notifies the target person 30 of an area 52 for patients having a fever when the body temperature of the target person 30 is higher than the predetermined threshold, and notifies the target person 30 of an area 54 for those without a fever when the body temperature is lower than the threshold. This enables the target persons 30 who have visited the fever clinic to be appropriately and automatically sorted to the area 52 and the area 54.

For example, the information processing system 100 transmits a notification content to a mobile communication terminal 300 of the target person 30, and causes the mobile communication terminal 300 to display the notification content. This ensures that the target person 30 grasps the notification content with respect to the target person 30. The association between the target person 30 and the mobile communication terminal 300 may be registered in advance. Any known method may be used as a method for the association. For example, the association between the target person 30 and the mobile communication terminal 300 is performed by a communication terminal arranged in a periphery of the image capturing apparatus 200 through communication with the mobile communication terminal 300 of the target person 30 using near field communication such as BLE (Bluetooth (registered trademark) Low Energy). For example, the association between the mobile communication terminal 300 and the target person 30 whose image is being captured by the image capturing apparatus 200 is performed by causing a display arranged in the periphery of the image capturing apparatus 200 to display code information such as a QR code (registered trademark) and causing the mobile communication terminal 300 to read the code information. Alternatively, for example, the association between the target person 30 and the mobile communication terminal 300 is performed by a reader arranged in the periphery of the image capturing apparatus 200 through reading of code information such as a QR code displayed on a display of the mobile communication terminal 300. The display of a notification content and the association between the target person 30 and the mobile communication terminal 300 may be realized by an application mounted on the mobile communication terminal 300.

The information processing system 100 may transmit a notification content to the display apparatus 400 arranged in the periphery of the target person 30, and cause the display apparatus 400 to display the notification content. This configuration allows the target person 30 to grasp the notification content, without associating the target person 30 and the mobile communication terminal 300 with each other. This configuration also allows people in the periphery of the target person 30 to be informed of a movement destination of the target person 30, which allows the people in the periphery to be aware of a wrong destination the target person 30 has arrived.

The information processing system 100 may also cause the notification content to be displayed via a web page relevant to the information processing system 100. The web page relevant to the information processing system 100 may be, for example, a web page created for the information processing system 100. Alternatively, the web page relevant to the information processing system 100 may be a web page created and operated by a management entity of the information processing system 100. The target person 30 can grasp the notification content by accessing the web page. Accordingly, the notification content can be grasped by the target person 30 without associating the target person 30 and the mobile communication terminal 300 with each other.

When the system 10 is applied to an isolation facility, the image capturing apparatus 200 is arranged at a front desk of the isolation facility, for example. Then, the image capturing apparatus 200 acquires target person information of the target person 30 who has come to the front desk, and transmits it to the information processing system 100.

The information processing system 100 may acquire, as well as receive the target person information from the image capturing apparatus 200, check-in information of the target person 30 from the front desk of the isolation facility. The check-in information may be transmitted to the information processing system 100 by a person in charge at the front desk. Further, when automatic check-in is adopted, for example, an acceptance terminal arranged at the front desk communicates with the mobile communication terminal 300 of the target person 30 to receive check-in information of the target person 30, and transmits the check-in information to the information processing system 100.

For example, based on the check-in information of the target person 30 and the body temperature of the target person 30, the information processing system 100 specifies a room as a movement destination of the target person 30 out of a plurality of rooms, and notifies the target person 30 of a room number indicating the specified room and key information for entering the room. The key information may be, for example, a passcode for unlocking a door of the room. For example, the information processing system 100 transmits the room number and the key information to the acceptance terminal, and causes the acceptance terminal to display them. Moreover, for example, the information processing system 100 transmits the room number and the key information to the mobile communication terminal 300 of the target person 30, and causes the mobile communication terminal 300 to display them.

When an electronic key is adopted in the isolation facility, the key information may be key data of the electronic key. For example, the information processing system 100 transmits the room number and the key data to the mobile communication terminal 300 of the target person 30.

The information processing system 100 may execute notification processing for notifying the target person 30 of a movement route that differs between a case where the body temperature of the target person 30 is higher than a predetermined threshold and a case where the body temperature is lower than the threshold. For example, the information processing system 100 notifies a movement route that uses a first elevator when the body temperature of the target person 30 is higher than the threshold, and notifies a movement route that uses a second elevator when the body temperature is lower than the threshold. Accordingly, it becomes possible to lower a possibility that a person having a high body temperature will use the same elevator as a person not having a high body temperature.

The information processing system 100 may receive captured images from other image capturing apparatuses arranged in a space that the target person 30 uses, to realize various managements.

When the system 10 is applied to facilities such as a school, kindergarten, and nursery school, the system 10 may recognize target persons staying at the facility by a face recognition technology or the like using the image capturing apparatus 200 arranged in the facility, sequentially acquire a body temperature of each target person, and accumulatively manage the body temperature of each target person by cooperating with a management system of the school, kindergarten, nursery school, community, or the like.

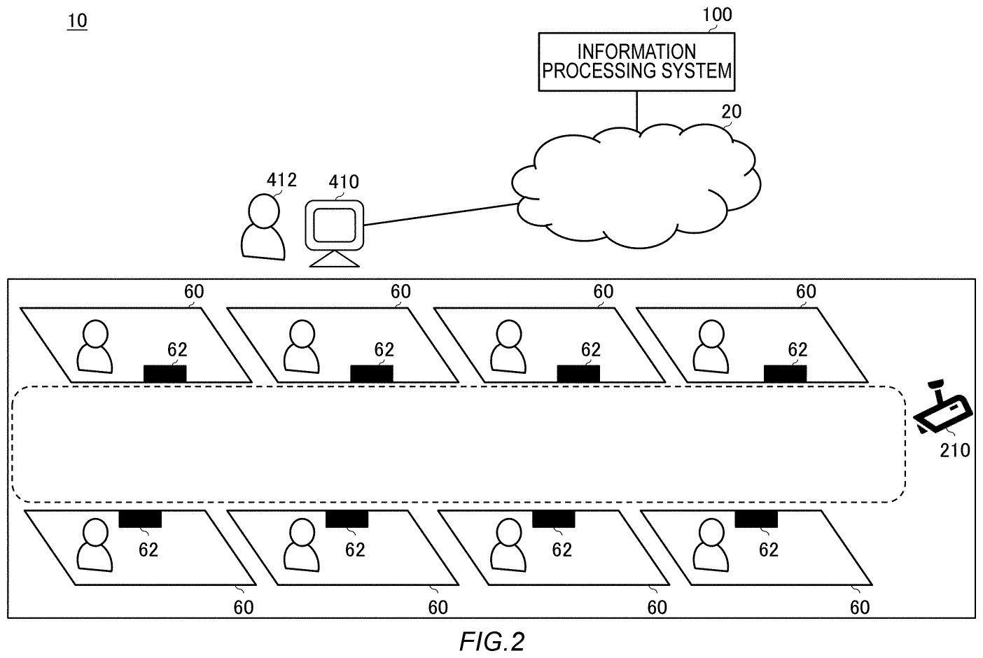

FIG. 2 schematically shows an example of the system 10. FIG. 2 exemplifies a case where the information processing system 100 receives, from an image capturing apparatus 210 arranged in a periphery of doors 62 of a plurality of rooms 60 of the isolation facility, a captured image obtained by capturing the doors 62 of the plurality of rooms 60.

The information processing system 100 may manage a situation of the plurality of rooms 60 by analyzing the captured image received from the image capturing apparatus 210. For example, the information processing system 100 manages an open/close situation of the plurality of doors 62. In addition, the information processing system 100 manages entering and exiting of people with respect to the plurality of rooms 60, for example.

The information processing system 100 may cooperate with a management system of the plurality of doors 62 to manage an unlocked/locked situation of the plurality of doors 62. The information processing system 100 may comprehensively manage the unlocked/locked situation of the plurality of doors 62, the open/close situation of the plurality of doors 62, and the entering and exiting of people with respect to the plurality of rooms 60. Then, the information processing system 100 may notify a predetermined destination when some kind of an anomaly is detected. For example, the information processing system 100 notifies a communication terminal 410 of an administrator 412 of the isolation facility. Accordingly, for example, it is possible to manage people who casually go out of the rooms 60, and the like. Further, by notifying the administrator 412 of the isolation facility, it is possible to manage entering and exiting timings with respect to the plurality of rooms 60, and thus reduce a chance of contact in a hallway or the like.

The information processing system 100 is not limited to the example shown in FIG. 2 and may receive a captured image from an image capturing apparatus arranged at any place. For example, the information processing system 100 receives, from an image capturing apparatus arranged in a no-entry area of the isolation facility or the like, a captured image of the no-entry area. For example, the information processing system 100 may analyze the captured image, and notify a predetermined destination when an entry of a person in the no-entry area is detected. Accordingly, it is possible to make the entering and exiting of people with respect to the no-entry area manageable. For example, the information processing system 100 may analyze the captured image, and when an entry of a person in the no-entry area is detected, perform warning by an alert tone using an apparatus that is installed near the no-entry area and generates sounds.

It is to be noted that the information processing system 100 may recognize a subject by using a face recognition technology or the like. For example, when an entry of a person in the no-entry area is detected, the information processing system 100 recognizes the entered person and determines whether that person has an entrance authority with respect to the area. A specific example of a person having an entrance authority is a medical staff and the like. The information processing system 100 does not notify when the person has the entrance authority and may notify a predetermined destination when the person does not have the entrance authority. When the person does not have the entrance authority, the information processing system 100 may perform warning by an alert tone using an apparatus that is installed near the no-entry area and generates sounds.

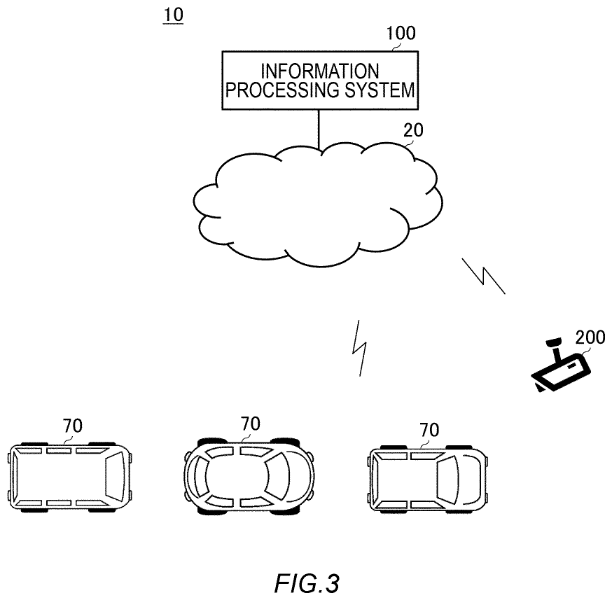

FIG. 3 schematically shows an example of the system 10. FIG. 3 exemplifies a case where the system 10 is applied to drive-through testing.

For example, a target person 30 on a vehicle 70 first uses the mobile communication terminal 300 to register a number of the vehicle 70 and people on the vehicle 70 in the information processing system 100. The registration may be performed via a web page relevant to the information processing system 100, or may be performed by a communication terminal arranged in the periphery of the image capturing apparatus 200 receiving information from the mobile communication terminal 300 and transmitting the information to the information processing system 100.

Next, the information processing system 100 receives, from the image capturing apparatus 200, target person information of each of the target persons 30 on the vehicle 70 and the number of the vehicle 70, that have been acquired by the image capturing apparatus 200. Then, the information processing system 100 registers the number of the vehicle 70, the information of the target person 30 on the vehicle 70, and the respective pieces of target person information in association with one another.

Accordingly, for example, guidance to a hospital to which the target person 30 is to be directed can be performed in accordance with the body temperature of the target person 30. In addition, the information processing system 100 can cooperate with a system that manages a congestion situation of each hospital to perform guidance to a hospital to which the target person 30 is to be directed in accordance with the congestion situation.

Further, for example, the vehicle 70 that the target person 30, whose body temperature is higher than a predetermined threshold, is on can be made manageable. As a specific example, it is possible to enable a destination of a visit of the vehicle 70 that the target person 30, whose body temperature is higher than the threshold, is on to be grasped as appropriate, so as to enable a situation where a person who is supposed to be heading to a hospital is headed toward a place other than the hospital, or the like to be manageable, for example.

Moreover, for example, when three people are on the vehicle 70 and a body temperature of one of them is higher than the threshold, the other two can be made manageable as a close contact person with respect to the person having a body temperature higher than the threshold.

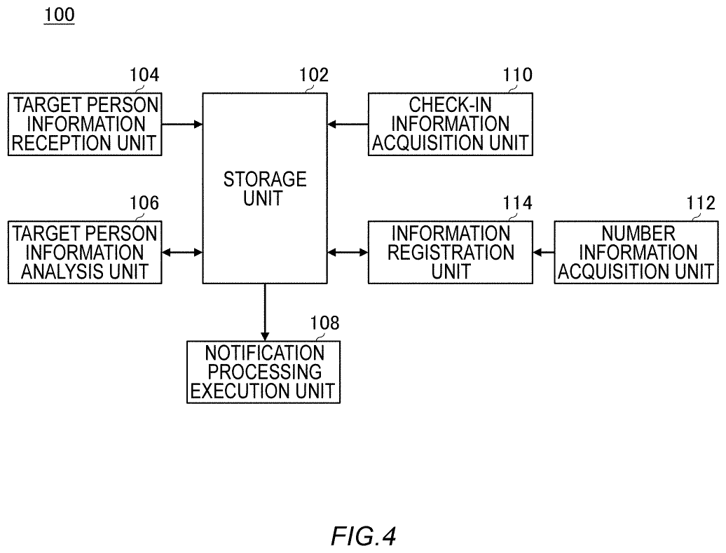

FIG. 4 schematically shows an example of a functional configuration of the information processing system 100. The information processing system 100 includes a storage unit 102, a target person information reception unit 104, a target person information analysis unit 106, a notification processing execution unit 108, a check-in information acquisition unit 110, a number information acquisition unit 112, and an information registration unit 114. It is to be noted that the information processing system 100 does not necessarily need to include all of these components.

The target person information reception unit 104 receives target person information including information related to a state of the target person 30 and a peripheral situation of the target person 30 from the image capturing apparatus 200 that has acquired the target person information. The target person information reception unit 104 stores the received target person information in the storage unit 102.

The target person information may include a body temperature of the target person 30. The target person information may include a body temperature of the target person 30 received by the image capturing apparatus 200 from a temperature sensor arranged in a periphery of the target person 30.

The target person information may include body motion information related to a body motion of the target person 30 specified by the image capturing apparatus 200 through an analysis of a captured image obtained by capturing of the target person 30. The body motion information may represent a level of breathing of the target person 30. The body motion information may represent a breathing speed of the target person 30. The body motion information may represent roughness of breathing of the target person 30. The body motion information may represent a part of a body that the target person 30 is holding.

The target person information may include a situation of people in the periphery of the target person 30 specified by the image capturing apparatus 200 through analysis of a captured image obtained by capturing of the periphery of the target person 30. The target person information may include the number of people in the periphery of the target person 30. The target person information may include the number of people positioned within a predetermined range from a position of the target person 30. The target person information may include a positional relationship between the target person 30 and the people in the periphery of the target person 30.

The target person information may include a carbon dioxide concentration in the periphery of the target person 30 received by the image capturing apparatus 200 from a carbon dioxide sensor arranged in the periphery of the target person 30. The target person information may include a carbon dioxide concentration per unit volume in a space in the periphery of the target person 30. The target person information may include an average value of carbon dioxide concentrations in a space within a predetermined range about the position of the target person 30.

The target person information may include an air volume in the periphery of the target person 30 received by the image capturing apparatus 200 from an air volume sensor arranged in the periphery of the target person 30. The target person information may include an air volume per unit volume in a space in the periphery of the target person 30. The target person information may include an average value of air volumes in a space within a predetermined range about the position of the target person 30.

The target person information reception unit 104 may receive, from the image capturing apparatus arranged in the periphery of the doors of the plurality of rooms 60, a captured image obtained by capturing the doors of the plurality of rooms 60. The target person information reception unit 104 may receive, from an image capturing apparatus arranged in a periphery of an area such as a no-entry area, a captured image of the area.

The target person information analysis unit 106 analyzes target person information received by the target person information reception unit 104. For example, the target person information analysis unit 106 derives an analysis result representing whether the body temperature of the target person 30 is higher than a predetermined threshold.

The target person information analysis unit 106 may derive an analysis result including physical condition information related to a physical condition of the target person 30 based on the body temperature of the target person 30 included in the target person information. The target person information analysis unit 106 may derive an analysis result including physical condition information related to a physical condition of the target person 30 based on the body motion information of the target person 30 included in the target person information. The physical condition information may represent whether a condition of the target person 30 is poor or not.

For example, when a level of breathing of the target person 30 is higher than a predetermined threshold, the target person information analysis unit 106 derives an analysis result including physical condition information representing that the condition of the target person 30 is poor. Alternatively, for example, when the breathing speed of the target person 30 is higher than a predetermined threshold, the target person information analysis unit 106 derives an analysis result including physical condition information representing that the condition of the target person 30 is poor. Alternatively, for example, when the roughness of breathing of the target person 30 is higher than a predetermined threshold, the target person information analysis unit 106 derives an analysis result including physical condition information representing that the condition of the target person 30 is poor.

The physical condition information may represent a part of the target person 30 where the condition is poor. For example, when the target person 30 is holding any part of the body, the target person information analysis unit 106 derives an analysis result including physical condition information representing that the part being held is the part of the target person 30 where the condition is poor.

The target person information analysis unit 106 may derive an analysis result representing a close-contact situation in the periphery of the target person 30 based on the situation of people in the periphery of the target person 30, that is included in the target person information. For example, the target person information analysis unit 106 derives an analysis result representing a close-contact situation that becomes higher as the number of people positioned within a predetermined range from the position of the target person 30 increases.

The target person information analysis unit 106 may derive an analysis result representing a crowded situation in the periphery of the target person 30 based on the carbon dioxide concentration in the periphery of the target person 30, that is included in the target person information. For example, the target person information analysis unit 106 derives an analysis result representing a crowded situation that becomes higher as the carbon dioxide concentration in the periphery of the target person 30 becomes higher.

The target person information analysis unit 106 may derive an analysis result representing a closed situation in the periphery of the target person 30 based on the air volume in the periphery of the target person 30, that is included in the target person information. For example, the target person information analysis unit 106 derives an analysis result representing a closed situation that becomes higher as the air volume in the periphery of the target person 30 becomes lower.

As described above, the target person information analysis unit 106 may derive analysis results representing the close-contact situation, the crowded situation, and the closed situation in the periphery of the target person 30 based on the target person information. Accordingly, a so-called three C's situation in a space captured by the image capturing apparatus 200 can be specified.

The target person information analysis unit 106 may analyze a captured image obtained by capturing the doors of the plurality of rooms 60 to analyze entering and exiting of people with respect to each of the plurality of rooms 60. The target person information analysis unit 106 may analyze a captured image of the area such as a no-entry area to analyze entering and exiting with respect to the area.

The notification processing execution unit 108 executes notification processing that is based on an analysis result obtained by the target person information analysis unit 106. For example, the notification processing execution unit 108 executes notification processing for notifying the target person 30 of a content that differs between a case where the body temperature of the target person 30 is higher than a predetermined threshold and a case where the body temperature is lower than the threshold.

For example, the notification processing execution unit 108 executes notification processing for notifying the target person 30 of a movement destination that differs between the case where the body temperature of the target person 30 is higher than the threshold and the case where the body temperature is lower than the threshold. Accordingly, the target person 30 having a high body temperature and the target person 30 not having a high body temperature can be guided to different places.

For example, the notification processing execution unit 108 executes notification processing for notifying the target person 30 of a movement route that differs between the case where the body temperature of the target person 30 is higher than the threshold and the case where the body temperature is lower than the threshold. Accordingly, the target person 30 having a high body temperature and the target person 30 not having a high body temperature can be guided to different movement routes, and thus a possibility of the former and the latter coming into contact with each other while moving can be reduced.

When the body temperature of the target person 30 is higher than the threshold, the notification processing execution unit 108 may execute notification processing with respect to a destination that has been pre-registered in association with the target person 30. For example, when the target person 30 is a student, the target person 30 himself/herself, a guardian of the target person 30, a teacher relevant to the target person 30, and the like can be registered as the destination. The teacher relevant to the target person 30 may be a homeroom teacher of the target person 30, for example. Alternatively, for example, when the target person 30 is a preschool child, a guardian of the target person 30, a teacher relevant to the target person 30, and the like can be registered as the destination.

As the notification processing, for example, the notification processing execution unit 108 transmits a notification content to the display apparatus 400 arranged in the periphery of the target person 30, and causes the display apparatus 400 to display the notification content. Further, as the notification processing, for example, the notification processing execution unit 108 transmits a notification content to the mobile communication terminal 300 owned by the target person 30, and causes the mobile communication terminal 300 to display the notification content. The notification processing execution unit 108 may cooperate with an application mounted on the mobile communication terminal 300 and cause the notification content to be displayed on a display of the mobile communication terminal 300, to thus notify the target person 30 of the notification content. As the notification processing, for example, the notification processing execution unit 108 causes the notification content to be displayed via a web page relevant to the information processing system 100. For example, the notification processing execution unit 108 may perform control to arrange the notification content in a predetermined region of a web page relevant to the information processing system 100.

The check-in information acquisition unit 110 acquires check-in information of the target person 30. For example, the check-in information acquisition unit 110 receives the check-in information of the target person 30 from the mobile communication terminal 300 of the target person 30. Alternatively, for example, the check-in information acquisition unit 110 receives the check-in information of the target person 30 from an acceptance terminal of a facility that the target person 30 is to enter.

The target person information analysis unit 106 may specify a room 60 as a movement destination of the target person 30 out of the plurality of rooms 60 based on the check-in information of the target person 30 acquired by the check-in information acquisition unit 110 and the target person information of the target person 30 received by the target person information reception unit 104. The notification processing execution unit 108 may transmit room information representing the room 60 specified by the target person information analysis unit 106 and key information for entering the room 60 to the mobile communication terminal 300 of the target person 30.

The key information may be a passcode for entering the room 60. The key information may be key data of an electronic key. The notification processing execution unit 108 may transmit the room information, the key information, and route information representing a movement route to the room determined depending on the body temperature of the target person 30, to the mobile communication terminal 300 of the target person 30. The route information may represent a movement route that differs between the case where the body temperature of the target person 30 is higher than the threshold and the case where the body temperature is lower than the threshold.

The notification processing execution unit 108 may execute notification processing that is based on an analysis result that is derived by the target person information analysis unit 106 and represents entering and exiting of people with respect to each of the plurality of rooms 60. For example, when casual entering and exiting of a person is detected, the notification processing execution unit 108 transmits notification information representing an alert to an administrator who is managing the plurality of rooms 60, or the like.

The notification processing execution unit 108 may execute notification processing that is based on an analysis result that is derived by the target person information analysis unit 106 and represents entering and exiting of people with respect to a no-entry area. For example, when an entry of a person in the no-entry area is detected, the notification processing execution unit 108 transmits notification information representing an alert to an administrator who is managing the no-entry area, or the like.

The number information acquisition unit 112 acquires vehicle number information of the vehicle 70 that the target person 30 is on. For example, the number information acquisition unit 112 receives the vehicle number information from the mobile communication terminal 300 owned by the target person 30. Alternatively, for example, the number information acquisition unit 112 receives the vehicle number information from the image capturing apparatus 200 that has acquired the vehicle number information of the vehicle 70 that the target person 30 is on. For example, the image capturing apparatus 200 analyzes a captured image obtained by capturing the vehicle 70 that the target person 30 is on, to acquire the vehicle number information of the vehicle 70.

The information registration unit 114 registers the vehicle number information of the vehicle 70 that the target person 30 is on and the analysis result of the target person information of the target person 30 in association with each other.

The notification processing execution unit 108 may execute notification processing for notifying a predetermined destination of the analysis result and the vehicle number information that have been registered in association with each other by the information registration unit 114. For example, the notification processing execution unit 108 executes notification processing for notifying a medical institution of the analysis result and the vehicle number information that have been registered in association with each other by the information registration unit 114.

The notification processing execution unit 108 may execute notification processing that is based on the physical condition information of the target person 30. For example, the notification processing execution unit 108 notifies a predetermined destination of the physical condition information of the target person 30. Examples of the predetermined destination may include the target person 30 himself/herself, related parties of the target person 30, an administrator of a facility that the target person 30 has visited, and the like, though the present invention is not limited to these.

The notification processing execution unit 108 may execute notification processing for notifying a pre-registered destination of a close-contact situation in the periphery of the target person 30. For example, the notification processing execution unit 108 transmits the close-contact situation in the periphery of the target person 30 to a destination registered in a service that notifies information of fever clinics, medical institutions, and the like at various locations. Accordingly, it is possible to cause a service registerer to grasp the close-contact situations at the various locations.

The notification processing execution unit 108 may execute notification processing for notifying a pre-registered destination of a crowded situation in the periphery of the target person 30. For example, the notification processing execution unit 108 transmits the crowded situation in the periphery of the target person 30 to a destination registered in a service that notifies information of fever clinics, medical institutions, and the like at various locations. Accordingly, it is possible to cause a service registerer to grasp the crowded situations at the various locations.

The notification processing execution unit 108 may execute notification processing for notifying a pre-registered destination of a closed situation in the periphery of the target person 30. For example, the notification processing execution unit 108 transmits the closed situation in the periphery of the target person 30 to a destination registered in a service that notifies information of fever clinics, medical institutions, and the like at various locations. Accordingly, it is possible to cause a service registerer to grasp the closed situations at the various locations.

The notification processing execution unit 108 may execute notification processing for notifying a pre-registered destination of the close-contact situation, the crowded situation, and the closed situation in the periphery of the target person 30. For example, the notification processing execution unit 108 transmits the three C's situation in the periphery of the target person 30 to a destination registered in a service that notifies information of fever clinics, medical institutions, and the like at various locations. Accordingly, it is possible to cause a service registerer to grasp the three C's situations at the various locations.

The notification processing execution unit 108 may execute notification processing performed by releasing the close-contact situation in the periphery of the target person 30 via a web page relevant to the information processing system 100. Accordingly, it is possible to cause any person who can view the web page to grasp the close-contact situations at the various locations. For example, by causing those who visit the fever clinics, medical institutions, and the like at the various locations to grasp the situations at the fever clinics, medical institutions, and the like at the various locations, leveling of medical consultations can be achieved.

The notification processing execution unit 108 may execute notification processing performed by releasing the crowded situation in the periphery of the target person 30 via a web page relevant to the information processing system 100. Accordingly, it is possible to cause any person who can view the web page to grasp the crowded situations at the various locations. For example, by causing those who visit the fever clinics, medical institutions, and the like at the various locations to grasp the situations at the fever clinics, medical institutions, and the like at the various locations, leveling of medical consultations can be achieved.

The notification processing execution unit 108 may execute notification processing performed by releasing the closed situation in the periphery of the target person 30 via a web page relevant to the information processing system 100. Accordingly, it is possible to cause any person who can view the web page to grasp the closed situations at the various locations. For example, by causing those who visit the fever clinics, medical institutions, and the like at the various locations to grasp the situations at the fever clinics, medical institutions, and the like at the various locations, leveling of medical consultations can be achieved.

The notification processing execution unit 108 may execute notification processing performed by releasing the close-contact situation, the crowded situation, and the closed situation in the periphery of the target person 30 via a web page relevant to the information processing system 100. Accordingly, it is possible to cause any person who can view the web page to grasp the three C's situations at the various locations. For example, by causing those who visit the fever clinics, medical institutions, and the like at the various locations to grasp the situations at the fever clinics, medical institutions, and the like at the various locations, leveling of medical consultations can be achieved.

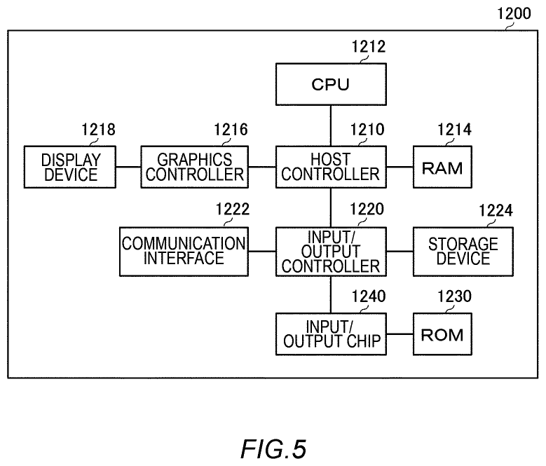

FIG. 5 schematically shows an example of a hardware configuration of a computer 1200 that functions as the information processing system 100. A program installed in the computer 1200 can cause the computer 1200 to function as one or more “units” of an apparatus according to the present embodiment, or cause the computer 1200 to perform operations associated with the apparatus or perform one or more “units” thereof according to the present embodiment, and/or cause the computer 1200 to perform the process according to the present embodiment or perform the steps of the process. Such a program may be executed by a CPU 1212 to cause the computer 1200 to perform specific operations associated with some or all of the blocks in the flow charts and block diagrams described in the specification.

The computer 1200 according to the present embodiment includes the CPU 1212, a RAM 1214, and a graphics controller 1216, which are connected to each other via a host controller 1210. The computer 1200 also includes input/output units such as a communication interface 1222, a storage device 1224, a DVD drive, and an IC card drive, which are connected to the host controller 1210 via an input/output controller 1220. The DVD drive may be a DVD-ROM drive, a DVD-RAM drive, and the like. The storage device 1224 may be a hard disk drive, a solid-state drive, and the like. The computer 1200 also includes a ROM 1230 and a legacy input/output unit such as a keyboard, which are connected to the input/output controller 1220 via an input/output chip 1240.

The CPU 1212 operates according to the programs stored in the ROM 1230 and the RAM 1214, thereby controlling each unit. The graphics controller 1216 acquires image data which is generated, by the CPU 1212, in a frame buffer or the like provided in the RAM 1214 or in itself so as to cause the image data to be displayed on a display device 1218.

The communication interface 1222 communicates with other electronic devices via a network. The storage device 1224 stores a program and data used by the CPU 1212 in the computer 1200. The DVD drive reads the program or the data from the DVD-ROM or the like, and provides the storage device 1224 with the program or the data. The IC card drive reads the program and data from the IC card, and/or writes the program and data to the IC card.

The ROM 1230 stores therein a boot program or the like executed by the computer 1200 at the time of activation, and/or a program depending on the hardware of the computer 1200. The input/output chip 1240 may also connect various input/output units via a USB port, a parallel port, a serial port, a keyboard port, a mouse port, or the like to the input/output controller 1220.

A program is provided by a computer-readable storage medium such as the DVD-ROM or the IC card. The program is read from the computer-readable storage medium, installed into the storage device 1224, RAM 1214, or ROM 1230, which are also examples of a computer-readable storage medium, and executed by the CPU 1212. The information processing described in these programs is read into the computer 1200, resulting in cooperation between a program and the above-described various types of hardware resources. An apparatus or method may be constituted by realizing the operation or processing of information in accordance with the usage of the computer 1200.

For example, when communication is performed between the computer 1200 and an external device, the CPU 1212 may execute a communication program loaded onto the RAM 1214 to instruct communication processing to the communication interface 1222, based on the processing described in the communication program. The communication interface 1222, under control of the CPU 1212, reads transmission data stored on a transmission buffer region provided in a recording medium such as the RAM 1214, the storage device 1224, the DVD-ROM, or the IC card, and transmits the read transmission data to a network or writes reception data received from a network to a reception buffer region or the like provided on the recording medium.

In addition, the CPU 1212 may cause all or a necessary portion of a file or a database to be read into the RAM 1214, the file or the database having been stored in an external recording medium such as the storage device 1224, the DVD drive (DVD-ROM), and the IC card, and perform various types of processing on the data on the RAM 1214. The CPU 1212 may then write back the processed data to the external recording medium.

Various types of programs, data, table, and various types of information such as a database may be stored in a recording medium to receive information processing. The CPU 1212 may perform various types of processing on the data read from the RAM 1214, which includes various types of operations, information processing, condition judging, conditional branch, unconditional branch, search/replacement of information, and the like, as described throughout this disclosure and designated by an instruction sequence of programs, and writes the result back to the RAM 1214. In addition, the CPU 1212 may search for information in a file, a database, and the like, in the recording medium. For example, when a plurality of entries, each having an attribute value of a first attribute associated with an attribute value of a second attribute, are stored in the recording medium, the CPU 1212 may search for an entry whose attribute value of the first attribute matches a designated condition, from among the plurality of entries, and read the attribute value of the second attribute stored in the entry, thereby acquiring the attribute value of the second attribute associated with the first attribute satisfying the predetermined condition.

The above-described program or software modules may be stored in the computer-readable storage medium on or near the computer 1200. In addition, a recording medium such as a hard disk or a RAM provided in a server system connected to a dedicated communication network or the Internet can be used as the computer-readable storage medium, thereby providing the program to the computer 1200 via the network.

Blocks in the flow charts and block diagrams in the present embodiment may represent steps of processes in which operations are performed or “units” of apparatuses responsible for performing operations. A specific step and “unit” may be implemented by a dedicated circuit, a programmable circuit supplied along with a computer-readable instruction stored on a computer-readable storage medium, and/or a processor supplied along with the computer-readable instruction stored on the computer-readable storage medium. The dedicated circuit may include a digital and/or analog hardware circuit, or may include an integrated circuit (IC) and/or a discrete circuit. The programmable circuit may include, for example, a reconfigurable hardware circuit including logical AND, logical OR, logical XOR, logical NAND, logical NOR, and other logical operations, and a flip-flop, a register, and a memory element, such as a field programmable gate array (FPGA) and a programmable logic array (PLA).

The computer-readable storage medium may include any tangible device capable of storing an instruction performed by an appropriate device, so that the computer-readable storage medium having the instruction stored thereon constitutes a product including an instruction that may be performed in order to provide means to perform an operation specified by the flow chart or block diagram. Examples of the computer-readable storage medium may include an electronic storage medium, a magnetic storage medium, an optical storage medium, an electromagnetic storage medium, a semiconductor storage medium, or the like. More specific examples of the computer-readable storage medium may include a floppy (registered trademark) disk, a diskette, a hard disk, a random access memory (RAM), a read only memory (ROM), an erasable programmable read only memory (EPROM or flash memory), an electrically erasable programmable read only memory (EEPROM), a static random access memory (SRAM), a compact disk read only memory (CD-ROM), a digital versatile disk (DVD), a Blu-ray (registered trademark) disk, a memory stick, an integrated circuit card, or the like.

The computer-readable instruction may include an assembler instruction, an instruction-set-architecture (ISA) instruction, a machine instruction, a machine dependent instruction, a microcode, a firmware instruction, state-setting data, or either source code or object code written in any combination of one or more programming languages including an object oriented programming language such as Smalltalk (registered trademark), JAVA (registered trademark), and C++, and a conventional procedural programming language such as a “C” programming language or a similar programming language.

The computer-readable instruction may be provided to a general-purpose computer, a special-purpose computer, or a processor or a programmable circuit of another programmable data processing device locally or via a local area network (LAN) or a wide area network (WAN) such as the Internet in order that the general-purpose computer, the special-purpose computer, or the processor or the programmable circuit of another programmable data processing device performs the computer-readable instruction to provide means to perform operations specified by the flow chart or the block diagram. Examples of the processor include a computer processor, a processing unit, a microprocessor, a digital signal processor, a controller, a microcontroller, or the like.

While the embodiments of the present invention have been described, the technical scope of the invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### EXPLANATION OF REFERENCES

10: system
20: network
30: target person
52: area
54: area
60: room
62: door
70: vehicle
100: information processing system
102: storage unit
104: target person information reception unit
106: target person information analysis unit
108: notification processing execution unit
110: check-in information acquisition unit
112: number information acquisition unit
114: information registration unit
200: image capturing apparatus
202: sensor
210: image capturing apparatus
300: mobile communication terminal
400: display apparatus
410: communication terminal
412: administrator
1200: computer
1210: host controller
1212: CPU
1214: RAM
1216: graphics controller
1218: display device
1220: input/output controller
1222: communication interface
1224: storage device
1230: ROM
1240: input/output chip

What is claimed is:

1. An information processing system, comprising:
a target person information reception unit configured to receive target person information including information related to a state of a target person and a situation in a periphery of the target person from an image capturing apparatus which has acquired the target person information;
a target person information analysis unit configured to analyze the target person information; and
a notification processing execution unit configured to execute notification processing based on an analysis result obtained by the target person information analysis unit.

2. The information processing system according to claim 1, wherein
the target person information includes a body temperature of the target person,
the target person information analysis unit is configured to derive an analysis result representing whether the body temperature of the target person is higher than a predetermined threshold, and
the notification processing execution unit is configured to execute notification processing for notifying the target person of a content which differs between a case where the body temperature of the target person is higher than the threshold and a case where the body temperature is lower than the threshold.

3. The information processing system according to claim 2, wherein
the notification processing execution unit is configured to execute notification processing for notifying the target person of a movement destination which differs between the case where the body temperature of the target person is higher than the threshold and the case where the body temperature is lower than the threshold.

4. The information processing system according to claim 2, wherein
the notification processing execution unit is configured to execute notification processing for notifying the target person of a movement route which differs between the case where the body temperature of the target person is higher than the threshold and the case where the body temperature is lower than the threshold.

5. The information processing system according to claim 1, wherein
the target person information includes a body temperature of the target person,
the target person information analysis unit is configured to derive an analysis result representing whether the body temperature of the target person is higher than a predetermined threshold, and
the notification processing execution unit is configured to execute notification processing with respect to a destination pre-registered in association with the target person when the body temperature of the target person is higher than the threshold.

6. The information processing system according to claim 2, wherein
the target person information includes the body temperature of the target person received by the image capturing apparatus from a temperature sensor arranged in the periphery of the target person.

7. The information processing system according to claim 2, comprising:
a check-in information acquisition unit configured to acquire check-in information of the target person,
wherein
the target person information analysis unit is configured to specify a room as a movement destination of the target person out of a plurality of rooms based on the check-in information and the body temperature of the target person, and
the notification processing execution unit is configured to transmit, to a mobile communication terminal of the target person, room information representing the room specified by the target person information analysis unit and key information for entering the room.

8. The information processing system according to claim 7, wherein
the key information is key data of an electronic key.

9. The information processing system according to claim 7, wherein
the notification processing execution unit is configured to transmit, to the mobile communication terminal, the room information, the key information, and route information representing a movement route to the room determined depending on the body temperature of the target person.

10. The information processing system according to claim 7, wherein
the target person information reception unit is configured to receive, from the image capturing apparatus arranged in a periphery of doors of the plurality of rooms, a captured image obtained by capturing the doors of the plurality of rooms,
the target person information analysis unit is configured to analyze entering and exiting of people with respect to each of the plurality of rooms, and
the notification processing execution unit is configured to execute the notification processing based on the analysis result obtained by the target person information analysis unit.

11. The information processing system according to claim 1, comprising:
a number information acquisition unit configured to acquire, from a mobile communication terminal owned by the target person, vehicle number information of a vehicle that the target person is on; and
an information registration unit configured to register the analysis result of the target person information and the vehicle number information in association with each other,
wherein the notification processing execution unit is configured to execute notification processing for notifying a predetermined destination of the analysis result and the vehicle number information which have been registered in association with each other by the information registration unit.

12. The information processing system according to claim 1, comprising:
a number information acquisition unit configured to acquire vehicle number information of a vehicle that the target person is on, from the image capturing apparatus which has acquired the vehicle number information; and
an information registration unit configured to register the analysis result of the target person information and the vehicle number information in association with each other.

13. The information processing system according to claim 1, wherein
the notification processing execution unit is configured to transmit a notification content to a display apparatus arranged in the periphery of the target person, and cause the display apparatus to display the notification content.

14. The information processing system according to claim 1, wherein
the notification processing execution unit is configured to transmit a notification content to a mobile communication terminal owned by the target person, and cause the mobile communication terminal to display the notification content.

15. The information processing system according to claim 1, wherein
the target person information includes body motion information related to a body motion of the target person specified by the image capturing apparatus through analysis of a captured image obtained by capturing of the target person,
the target person information analysis unit is configured to derive an analysis result including physical condition information related to a physical condition of the target person, and
the notification processing execution unit is configured to execute notification processing based on the physical condition information of the target person.

16. The information processing system according to claim 1, wherein
the target person information includes a situation of people in the periphery of the target person specified by the image capturing apparatus through analysis of a captured image obtained by capturing of the periphery of the target person,
the target person information analysis unit is configured to derive an analysis result representing a close-contact situation in the periphery of the target person, and
the notification processing execution unit is configured to execute notification processing for notifying a pre-registered destination of the close-contact situation in the periphery of the target person.

17. The information processing system according to claim 1, wherein
the target person information includes a carbon dioxide concentration in the periphery of the target person received by the image capturing apparatus from a carbon dioxide sensor arranged in the periphery of the target person,
the target person information analysis unit is configured to derive an analysis result representing a crowded situation in the periphery of the target person, and
the notification processing execution unit is configured to execute notification processing for notifying a pre-registered destination of the crowded situation in the periphery of the target person.

18. The information processing system according to claim 1, wherein
the target person information includes an air volume in the periphery of the target person received by the image capturing apparatus from an air volume sensor arranged in the periphery of the target person,
the target person information analysis unit is configured to derive an analysis result representing a closed situation in the periphery of the target person, and
the notification processing execution unit is configured to execute notification processing for notifying a pre-registered destination of the closed situation in the periphery of the target person.

19. The information processing system according to claim 1, wherein
the target person information includes a situation of people in the periphery of the target person specified by the image capturing apparatus through analysis of a captured image obtained by capturing of the periphery of the target person,
the target person information analysis unit is configured to derive an analysis result representing a close-contact situation in the periphery of the target person, and
the notification processing execution unit is configured to execute notification processing performed by releasing the close-contact situation in the periphery of the target person via a web page relevant to the information processing system.

20. The information processing system according to claim 1, wherein
the target person information includes a carbon dioxide concentration in the periphery of the target person received by the image capturing apparatus from a carbon dioxide sensor arranged in the periphery of the target person,
the target person information analysis unit is configured to derive an analysis result representing a crowded situation in the periphery of the target person, and
the notification processing execution unit is configured to execute notification processing performed by releasing the crowded situation in the periphery of the target person via a web page relevant to the information processing system.

21. The information processing system according to claim 1, wherein
the target person information includes an air volume in the periphery of the target person received by the image capturing apparatus from an air volume sensor arranged in the periphery of the target person,
the target person information analysis unit is configured to derive an analysis result representing a closed situation in the periphery of the target person, and
the notification processing execution unit is configured to execute notification processing performed by releasing the closed situation in the periphery of the target person via a web page relevant to the information processing system.

22. The information processing system according to claim 1, further comprising the image capturing apparatus.

23. A computer-readable storage medium having stored thereon a program for causing a computer to function as:
a target person information reception unit configured to receive target person information including information related to a state of a target person and a situation in a periphery of the target person from an image capturing apparatus which has acquired the target person information;
a target person information analysis unit configured to analyze the target person information; and
a notification processing execution unit configured to execute notification processing based on an analysis result obtained by the target person information analysis unit.

24. An information processing method executed by a computer, comprising:
receiving target person information including information related to a state of a target person and a situation in a periphery of the target person from an image capturing apparatus which has acquired the target person information;
analyzing the target person information; and
executing notification processing based on an analysis result obtained in the analyzing of the target person information.
